(19) United States
(12) Patent Application Publication
(10) Pub. No.: US 2026/0198834 A1
(43) Pub. Date: Jul. 16, 2026

(54) MEDICAL DEVICE FOR MONITORING BIOLOGICAL PARAMETERS

(71) Applicant: HOME HABILIS, Beauvais (FR)

(72) Inventor: Julien Gautier, Beauvais (FR)

(73) Assignee: HOME HABILIS, Beauvais (FR)

(21) Appl. No.: 18/866,585

(22) PCT Filed: May 17, 2023
(86) PCT No.: PCT/EP2023/063411

§ 371 Date: Nov. 18, 2024

(30) Foreign Application Priority Data
May 18, 2022 (FR) FR2204694

(51) Int. Cl. A61B 5/33; A61B 5/0205; A61B 5/0535; A61B 5/0537

(52) U.S. Cl. CPC A61B 5/33; A61B 5/0205; A61B 5/0535; A61B 5/0537

(57) ABSTRACT

The medical device (10) for monitoring biological parameters of a user, includes a plate (1) for measuring a mass of the user, at least a first electrode and a second electrode (11, 12, 21, 22) for performing impedance measurements, and a device for measuring blood pressure and/or heart rate (23).

The invention relates to a medical device for monitoring biological parameters.

The daily monitoring of water and salts (phosphorus, calcium, potassium, sodium chloride) intakes is of great importance to patients living with chronic kidney disease. Specifically, poor management of such intakes is detrimental to the ability of the patient to tolerate dialysis (notably by causing intra-dialysis hypotension and/or cramps) and greatly increases the probability of the onset of side effects associated with the disease, such as hypertension and/or cardiovascular problems.

Only a high level of deprivation, particularly with regard to water and salts, allows patients with chronic kidney disease to live with their disease. It is therefore of the greatest importance to provide them with means for regularly, notably daily, monitoring their intake of liquid derived from their food, thus enabling them to improve their tolerance to dialysis and reduce the risk of the onset of side effects of the dialysis.

In current practice, daily monitoring of water intake is performed empirically and approximately simply by weighing and by comparing the measured weight against a reference weight referred to as the “ideal weight” of the patient. The reference weight of the patient is also referred to as their “dry weight” because it corresponds to an estimate of the weight that the patient should normally attain at the end of a dialysis session when all of the excess fluid has been removed. The dry weight of a patient who has undergone dialysis is generally determined empirically by the care team as being the lowest weight that the patient can tolerate and that does not generate any undesirable effects in the patient. It is a compromise, reviewed by consultation, between clinical observation of relatively observable symptoms, history of intolerance during dialysis, and measured weight and blood pressure. Thus, in most cases, the dry weight is estimated by an iterative approach, errors in the estimate of dry weight being detectable either through the development of undesirable effects during dialysis sessions (if the dry weight has been underestimated) or by the onset of water retention symptoms (if the dry weight has been overestimated).

An assessment of the body composition would advantageously make it possible to distinguish between water mass and total mass and thus make it possible to determine a more reliable dry weight to which to refer in order to manage the quantity of fluid that a patient absorbs between two dialysis sessions, and thus determine the volume of water to be removed during a dialysis session.

In addition, daily at-home monitoring of indicators relating to the side effects of the disease would allow an improvement in reaction time when actively responding to these side effects, particularly in actively responding to hypertension and cardiovascular problems.

There are currently devices that allow body composition to be assessed, notably in the home. However, these devices are generally ill-suited to the body composition and level of physical activity of a patient presenting with chronic kidney disease, notably given that the median age for a patient treated by dialysis is 71 years of age and because the disease is almost systematically associated with multiple comorbidities. In addition, the existing devices do not take into consideration the specific needs of these patients, such as the need to detect and monitor the side effects of the disease.

Moreover, the various devices that measure the body composition of patients treated by dialysis yield results that differ significantly according to the device used. For this reason, it is not possible, for the one same patient, to compare measurements originating from different devices. As a result, it is not possible to monitor a patient on the basis of data originating from different devices.

It is an object of the invention to provide a device for monitoring biological parameters that overcomes the above disadvantages and improves on the devices known from the prior art.

In particular, it is an object of the invention to produce a device that is simple and reliable and that allows daily monitoring of the weight of the patient and/or of the quantity of fluid absorbed between two dialysis sessions and/or daily monitoring of the side effects of their chronic kidney disease.

To this end, the invention relates to a medical device for monitoring biological parameters of a user, comprising:
a plate for measuring the user's mass,
at least a first and a second electrode for performing impedance measurements, and
a device for measuring blood pressure and/or heart rate.

In one embodiment, the device for measuring blood pressure and/or heart rate is a photoplethysmographic sensor also capable of measuring the oxygen saturation of the user's blood.

In one embodiment, the plate is equipped with the first electrode able to measure impedance at one of the user's feet, and the second electrode is positioned in such a way as to be able to measure impedance at the user's first hand.

In one embodiment, the medical device comprises a third electrode positioned in contact with the user's second hand, and the second and third electrodes are able together to record an electrocardiogram between the user's first hand and second hand.

In one embodiment, the medical device comprises a temperature sensor making it possible to measure the user's skin temperature.

In one embodiment, the medical device comprises a means for adjusting the height of the second electrode relative to the plate so as to adapt a vertical distance, measured between the second electrode and the plate, to suit the user's height.

In one embodiment, the medical device comprises a first handgrip, on which the second electrode is fixed, arranged on a rigid arm substantially parallel to the plate, and the adjustable means is a system for adjusting the height of said rigid arm comprising the first handgrip relative to the plate to enable adjustment of the height of the first handgrip relative to the plate.

In one embodiment, electrodes of the medical device are suited to measuring impedance at a user's foot or hand by injecting a current with a strength comprised between 8 and 200 μA, or between 8 and 150 μA or between 8 and 100 μA or even of around 32 μA, give or take 10%, and with a frequency sweep extending from 125 Hz to 500 kHz, or even up to 800 kHz, or even extending between 4 kHz and 300 KHz.

In one embodiment, the medical device comprises an electronic processing unit. The electrodes and/or the device for measuring blood pressure and/or heart rate, and/or the temperature sensor are connected to the electronic processing unit by a wired or wireless communication device so that the impedance measurements taken using the electrodes are able to be transmitted to the electronic processing unit and so that the electronic processing unit is able to control the current transmitted by the electrodes to a user's hand or foot.

In one embodiment, the medical device comprises hardware elements and/or software elements configured for performing the following processing operations:
measuring a user's total mass,
measuring a foot-hand impedance,
measuring a volume of intracellular water contained in a user's body,
measuring a volume of extracellular water contained in a user's body,
measuring a total volume of water contained in a user's body,
measuring a dry weight,
measuring a hyperhydration volume,
and optionally measuring a variation in dry weight,
and optionally measuring a variation in hyperhydration,
and optionally measuring a blood pressure,
and optionally measuring a pulse wave,
and optionally measuring a heart rate,
and optionally measuring a blood oxygen saturation level,
and optionally measuring a hand-hand electrocardiogram,
and optionally measuring a skin temperature.

In one embodiment, the measurement of a volume of intracellular water, the measurement of a volume of extracellular water, and the measurement of a total volume of water are performed independently of one another.

In one embodiment, the medical device comprises a human-machine interface enabling the displaying of at least one item of data selected from among:
a user's total mass,
a total volume of water contained in a user's body,
a user's dry weight,
a variation in the user's dry weight,
a hyperhydration volume,
a variation in hyperhydration,
a user's heart rate,
a user's blood pressure,
a user's skin temperature,
a visual indication of bioimpedance.

The attached drawings depict, by way of example, one embodiment of a monitoring device according to the invention.

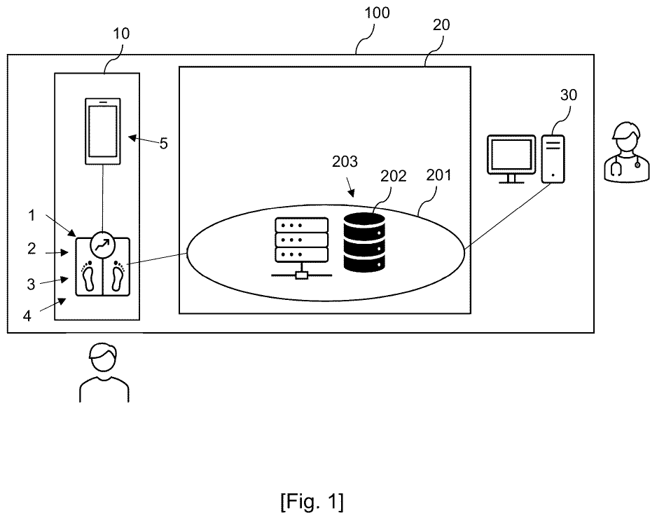

FIG. 1 depicts a monitoring system equipped with a medical monitoring device according to the invention.

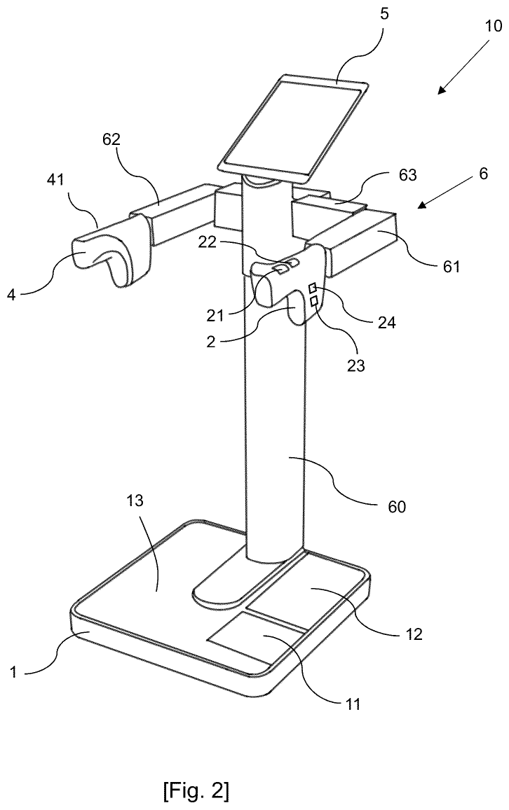

FIG. 2 depicts one embodiment of a medical monitoring device according to the invention.

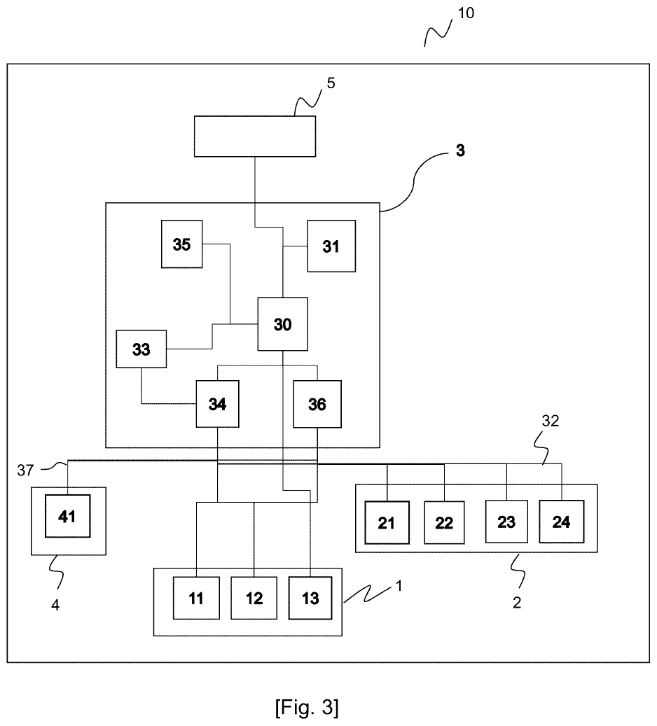

FIG. 3 schematically depicts an electronic processing unit of the medical monitoring device according to the embodiment of the invention.

One example of a system for monitoring biological parameters which is equipped with a monitoring device according to the invention, is described hereinafter with reference to FIGS. 1 to 3.

In the remainder of the document, the term “weight” is to be understood as meaning a mass measured in kilograms.

The medical monitoring system 100 chiefly comprises the following elements:
at least one medical monitoring device 10,
a distributed network infrastructure 20, and
at least one terminal 30 for a healthcare professional.

The distributed network infrastructure 20 comprises a network 201, a centralized memory 202 housing a database 203 and programs for processing the data contained in the database. The centralized memory also houses programs that manage the communications between a control unit 3 of each of the at least one medical monitoring device 10 and the user terminals 30 assigned to the healthcare professionals.

Such an embodiment, employing a distributed architecture, enables the simplest calculations to be performed directly in the medical device 10, and the more complex calculations to be delegated to a computer server connected to the database 203.

In a minimalist embodiment, the medical monitoring system 100 could be made up of just one medical device 10. The calculations would then all be performed locally, namely in the medical device 10.

In the remainder of the document, the medical monitoring device 10 is also referred to as “medical device 10” or “device 10”.

The medical device 10, particularly depicted in FIG. 2, comprises:
a plate 1 for measuring the user's mass,
at least a first and a second electrode 11, 12, 21, 22 for performing impedance measurements at least at two distinct frequencies, and for measuring at least a volume of water in the user's body, and
a device 23 for measuring blood pressure and/or heart rate.

The water volume measured by the first and second electrode 11, 12, 21, 22 may be the volume of intracellular water, the volume of extracellular water, and the total volume of water. These measurements are described later on in the document.

The characteristics, notably the frequency characteristics, of a current injected into the electrodes in order to measure impedance are detailed later on in this document. The device uses at least two distinct current frequencies: a first frequency for measuring a volume of extracellular water in a body, and a second frequency, higher than the first frequency, for measuring a total volume of water in the body. Advantageously, the frequency sweep of a current injected into the electrodes comprises at least two, or even three, frequencies (at least a low frequency and/or medium frequency, and a high frequency), or even at least six frequencies, or even at least twelve frequencies. What is meant by a high frequency is a frequency higher than 50 kHz. What is meant by a low frequency is a frequency lower than 5 kHz. What is meant by a medium frequency is a frequency comprised between 5 and 50 KHz.

In one embodiment, the plate 1 is equipped with at least one load cell 13 for measuring the user's total mass. Advantageously, the plate is equipped with several load cells 13, for example four load cells 13, to optimize the precision with which the total mass of the user is measured.

In one embodiment, the plate 1 is equipped with the first electrode 11, 12 able to measure impedance at one of the user's feet. Furthermore, the second electrode 21, 22 is positioned in such a way as to be able to measure impedance at the user's first hand.

The first and second electrodes are thus able together to measure impedance between a user's foot and hand, notably to measure impedance between a foot and a hand on the same side of a user's body, namely a foot and a hand situated in the same hemicorpus of the user's body. As a preference, in order to limit the extent to which cardiac operation interferes with the impedance measurements, the measurements are taken in the right hemicorpus.

In the remainder of this document, the impedance measurements between a user's foot and hand are referred to as “foot-hand impedance measurements”.

Note that what is meant by an impedance measurement is any direct and indirect measurement that makes it possible to obtain such a parameter directly, such as measurements of voltage, of variations in voltage as a function of time, of current and/or variations in current as a function of time, or derivatives thereof.

More generally, in the remainder of the document, the term “measurement” may denote data originating directly from the sensors, and/or data calculated from data collected from the sensors. The calculations may include algorithmic calculations, notably calculations involving learning algorithms, for example neural network learning calculations.

Moreover, the term “foot” is used to denote a body part that may encompass a foot, notably the sole of the foot, the top of the foot and the toes, and that may even extend above the ankle. Likewise, the term “hand” is used to denote a body part that may encompass a hand, notably the palm of the hand, the back of the hand and the fingers, and that may extend to above the wrist.

Advantageously, the plate 1 may comprise more than one electrode, for example two electrodes 11, 12, arranged in such a way as to be simultaneously in contact with a foot of a user standing upright on the plate. For example, one electrode 11 may be in contact with the heel of the foot while another electrode 12 is in contact with the front of the foot. In the remainder of the document, the term “first electrode” refers to one or more than one electrode positioned on the plate 1. The first electrode is intended to measure impedance between one of the user's feet and another region of the user's body.

In addition or as an alternative, at least two electrodes 21, 22 may be positioned at a user's first hand. For example, one electrode 21 may be in contact with the palm of the first hand and another electrode 22 may be in contact with a digit of the first hand. In the remainder of the document, the term “second electrode” refers to one or more than one electrode positioned in contact with a user's first hand and intended to take measurements at the user's first hand.

Thus, depending on the number of electrodes comprised in the medical device 10, the first and second electrodes may make it possible to take different types of impedance measurements between a foot and first a hand of the user, for example bipolar, tripolar or quadripolar measurements.

The medical device 10 further comprises a device 23 for measuring blood pressure and/or heart rate.

In one embodiment, the device for measuring blood pressure and/or heart rate is a photoplethysmographic sensor 23.

The photoplethysmographic sensor 23 is able to take all or some of the following measurements: the user's blood pressure, heart rate and blood oxygen saturation.

The photoplethysmographic sensor 23 may include an array of at least two emitting LEDs and one or two receiving photodiodes. The at least two emitting LEDs produce light at two different wavelengths, the respective wavelengths for these LEDs being able for example to lie between 500 and 750 nm in the case of the first, and between 850 and 1000 nm in the case of the second.

Other embodiments of a device 23 for measuring blood pressure are conceivable, for example a blood pressure cuff.

The medical device may further comprise a third electrode 41 arranged in contact with the user's second hand. In the remainder of the document, the term “third electrode” refers to one or more than one electrode positioned in contact with a user's second hand and intended to take measurements at the user's second hand.

Advantageously, the second and third electrodes 21, 22, 41 are able together to record an electrocardiogram between the user's first hand and second hand.

In the remainder of this document, the impedance measurements between a user's first and second hand are referred to as “hand-hand impedance measurements”.

In addition, the second and third electrodes 21, 22, 41 are able together
to measure heart rhythm, and/or
to measure a pulse wave traveling between the second and third electrodes 21, 22, 41, and/or
to measure a velocity of the pulse wave traveling between the second and third electrodes 21, 22, 41.

The medical device may further comprise a temperature sensor 24 making it possible to measure the user's skin temperature.

Advantageously, the medical device comprises a means for adjusting the height of the second electrode 21, 22 relative to the plate 1 so as to adapt a vertical distance, measured between the second electrode and the plate, to suit the user's height. Thus, the second electrode 21, 22 may be positioned in such a way as to suit a natural position of a hand which would then be placed in contact with the second electrode 21, 22.

The means of adjustment may adopt various forms. For example, in one preferred embodiment, the device 10 may comprise a first handgrip 2 on which the second electrode 21, 22 is fixed. The adjusting means may then be a system for adjusting the height of the first handgrip 2 relative to the plate 1.

The medical device 10 may further comprise a second handgrip 4 equipped with the third electrode 41. The adjusting means may also make it possible to adjust the height of the second handgrip relative to the plate 1.

According to the embodiment, the medical device therefore comprises a plate to receive a user's feet, and two handgrips for respectively receiving the user's hands or even arms. Advantageously, these handgrips are rigid and are arranged on two rigid arms, as will be detailed later on.

The device 23 for measuring blood pressure and/or heart rate, and/or the temperature sensor 24 may be positioned on the first or the second handgrip 2, 4.

The medical device 10 additionally comprises an electronic processing unit 3, particularly depicted in FIG. 3. This unit incorporates at least one computer 30 and connections 32 enabling the computer 30 to be connected to sensors 13, 23, 24 and electrodes 11, 12, 21, 22, 41 of the medical device 10. The computer 30 may thus receive the data measured by the sensors. The electronic processing unit 3 additionally comprises a medium on which to record data or electronic memory 31. It also comprises a power source 34, connected to all or some of the electrodes, thus enabling electrical current to be injected via the electrodes. It additionally comprises at least one voltage sensor 36 able to collect physiological data via the measurement electrodes. This voltage sensor 36 may, as an alternative, be located at a measurement electrode, or remotely, within the electronic processing unit 3, as depicted. Finally, the electronic processing unit 3 comprises a communication device 35 enabling it to transmit electronic data, such as for example body-composition measurements or a blood pressure, to the outside and/or to receive data, such as setup data or messages sent by a healthcare professional, from the outside. The electronic memory 31 also enables storage of the measurements and/or the data calculated locally in the medical device 10. The measurements and/or the data calculated locally in the medical device 10 are then transmitted to the database 203 via the network 20. The measurements and/or the data calculated locally in the medical device 10 are then advantageously saved in the memory 31 for a minimal length of time, notably if the network 20 is unavailable. The minimal length of time for which the data is saved may for example be 15 days.

The data in the electronic memory 31 can be read by the computer 30 or even by any other computing device of the electronic processing unit 3 on which is recorded a computer program for operating the medical device 10, notably for performing the measurements and calculations of biological parameters that are described in the remainder of this document.

In one embodiment depicted in FIG. 2, the medical device 10 comprises a support 6 connected to the plate 1 and to the first and second handgrips 2, 4. The support 6 comprises a vertical post 60 fixed at its lower part to the plate 1. The vertical post 60 is preferably perpendicular to the plate 1. It is positioned in such a way as to allow a user to step onto the plate 1 and position one of their feet on the two electrodes 11, 12 of the plate simultaneously.

The support 6 also comprises a U-shaped frame made up of three parallelepipedal portions 61, 62, 63 joined together, a central portion 63 being interposed between two portions 61, 62 that form the respective two branches of the U. In one embodiment, the portions 61, 62, 63 are joined together rigidly and the assembly that they form extends in a substantially horizontal plane, which is to say a plane parallel to the plane of the plate 1. Alternatively, the portions that form the branches 61, 62 are able to rotate about the central portion 63. The branch 61 comprises a first end connected to the central portion 63 and a second end connected to the first handgrip 2. The branch 62 comprises a first end connected to the central portion 63 and a second end connected to the second handgrip 4. The central portion 63 is fixed perpendicular to the vertical post 60 in an upper region of the vertical post 60. In one advantageous embodiment, the fixing between the vertical post 60 and the central portion 63 comprises a means for the heightwise adjustment of the U-shaped frame. Heightwise adjustment of the U-shaped frame advantageously allows the height of the handgrips 2, 4, namely the vertical distance separating the handgrips 2, 4 from the plate 1, to be adjusted.

According to one embodiment, the two branches 61, 62 of the medical device 10 take the form of two rigid arms.

The two branches 61, 62 comprise the electrode(s) intended to collaborate with a user's hand, via a handgrip 2 or otherwise.

As a variant, the rigid frame positioned at hand height so as to collaborate with a user's hands, may have a shape other than a U-shape.

In addition, the height-adjustment means may comprise a locking/unlocking device allowing the two branches 61, 62 to be fixed or not fixed relative to the support 6, thus enabling them to be kept at a chosen height. This locking/unlocking device that also allows this position to be unlocked so that they can be moved and their height adjusted. For that, a guide device may allow them to be moved, for example using at least one guide rail, notably to be moved in vertical translation. The support 6 may for example slide in vertical translation for adjusting it, then may be kept at a chosen height by a mechanism involving pins inserted into a certain respective opening in the support and in the vertical post, corresponding to a chosen height for the support.

Thus, the device ensures that the electrodes are adjusted to the correct height for collaborating with a user's hand(s), then guarantees that this height is kept constant during regular, for example daily, measurements for optimal mutual consistency between the various measurements.

The vertical post and/or the U-shaped frame are preferably hollow so as to be able to incorporate the connections 32 for the sensors 11, 12, 13, 21, 22, 23, 24, 41 and possibly the electronic processing unit 3.

The electronic processing unit 3 may be arranged actually within the medical device 10. As a variant, it takes the form of a unit remote from the plate 1 and from the first and second handgrips 2, 3 but connected to these two elements by a wired or wireless communications device 37, as depicted in FIG. 3.

In one embodiment, the electronic processing unit 3 has a distributed architecture.

Notably, in this embodiment, the management of the medical device (including the processing of the data and the control of the sensors and electrodes) is distributed between a plurality of microcontrollers each at least incorporating a computer and a recording medium. At least two local microcontrollers are for example respectively positioned at each handgrip 2, 4. Each local microcontroller is thus situated near the electrodes 21, 22 and 41.

A central microcontroller has the task of orchestrating the sequence of measurements by coordinating the local microcontrollers and collecting the measurements taken by the local microcontrollers. As a variant, there may be just one local microcontroller near the electrodes, in communication with a central microcontroller. This set comprising one or more local microcontroller(s), a central microcontroller and their associated electronic memories forms a solution equivalent to the computer 30 and electronic memory 31 described hereinabove.

Each local microcontroller is advantageously positioned as close as possible to the sensors that it operates. The local microcontrollers each incorporate a multiplexer 33, an electrical power source 34 and a voltmeter 36 that enable them each to control the bioimpedance electrodes. Thus, a local electronic processing unit similar to the one described with reference to FIG. 3 is positioned as close as possible to the sensors of the device. Such an arrangement makes it possible to improve the quality of the analog measurements by minimizing the signal attenuation and the noise.

A local microcontroller is also advantageously positioned as close as possible to the photoplethysmographic sensor 23, emitting LEDs and receiving photodiodes in order to control same.

The central microprocessor also manages the communications with a human-machine interface 5 assigned to a user of the medical device 10, notably to a patient living with chronic kidney disease. The human-machine interface 5 forms part of the medical device 10.

The central microcontroller moreover manages communications with the network 20, for transmitting data originating from the medical device 10 to the remote database 203.

In one embodiment, the central microcontroller manages communications via the network 20 with a remote computer 30, or any portable object, on which there may be a human-machine interface to allow user of the monitoring system 100, notably a healthcare professional, to consult the data originating from the medical device and/or to manage the settings in the medical device 10.

The invention also relates to a computer-readable recording medium comprising instructions which, when executed by a computer, cause the latter to implement the method for calculating a water volume, as well as calculations for estimating biological parameters, such as blood pressure, vascular stiffness, heart rhythm and oxygen saturation.

The electronic processing unit 3 may thus control the operation of the medical device 10 by instructing it to measure bioimpedance between a patient's foot and hand. To do that, the electrical power source 34 may generate an electric current via the electrodes 11, 12, 21 and 22, and then measure induced electrical data, notably using the voltage sensor 36. The voltage induced by the passage of the current is measured at least at one measurement electrode by the voltage sensor 36 and an application of Ohm's law makes it possible to determine the bioelectric impedance of the environment through which the current has passed, namely of a patient's body.

To complement this, in one embodiment, the medical examination device 10 could be capable of measuring impedance in a region including the patient's cardiac region. To do that, a current is generated between the electrodes 21, 22, 41 of the first and second hands, and measurements can be used to deduce therefrom the bioelectric impedance of the environment through which the current has passed, namely the cardiac region. In this embodiment, the third electrode would advantageously comprise several electrodes arranged in contact with the second hand.

In one mode of use, the electrical power source 34 injects a low-strength current of a strength in a range from 8 to 200 μA, or from 8 to 150 μA or from 8 to 100 μA—for example of around 32 μA, give or take 10%—with a chosen frequency sweep which may extend from 125 Hz to 500 kHz, or even from 125 Hz to 800 KHz. As a preference, the frequency sweep will extend from 4 kHz to 300 kHz. This frequency sweep will make it possible, per frequency zone, to pass through the various types of cell membrane and explore the extracellular and intracellular zones. Alternatively, the strength of the current injected by the electrical power source 34 could lie in a broader range of values, for example in a range of values from 8 to 200 μA.

Through its electrical measurements, the medical device 10 performs bioimpedance—or frequency-based impedance—spectroscopy, which provides a measurement of the volume of water in the body.

The medical device 10 comprises hardware elements 1, 2, 3, 4, 5, 11, 12, 13, 21, 22, 23, 24, 41 and/or software elements configured for performing all or some of the following processing operations:
measuring a user's total mass,
measuring a foot-hand impedance,
measuring a volume Vᵢ of intracellular water contained in a user's body,
measuring a volume Vₑ of extracellular water contained in a user's body,
measuring a total volume Vₜ of water contained in a user's body,
measuring a dry weight,
measuring a hyperhydration volume,
and optionally measuring a variation in dry weight,
and optionally measuring a variation in hyperhydration,
and optionally measuring a blood pressure,
and optionally measuring a pulse wave,
and optionally measuring a heart rate,
and optionally measuring a blood oxygen saturation level,
and optionally measuring a hand-hand electrocardiogram,
and optionally measuring a skin temperature.

The user's total mass, expressed in kilograms, can be determined by measurements originating from the at least one load cell. To complement this or alternatively, the foot-hand impedance measurement may be used for determining the user's total weight. As a preference, the total mass is measured in a range from 0 to 150 kilograms, or even in a range from 0 to 200 kilograms, with a precision at least to within 0.5 kilogram or even to within at least 0.1 kilogram.

The foot-hand impedance measurements are performed by means of the electrodes 11, 12, 21 and 22 which can inject an electrical current between one of the user's feet and one of their hands and can then measure the induced electrical data.

Analysis of the electrical data originating from the foot-hand impedance measurements makes it possible to calculate the volume, in liters, of water contained in the user's body. This water volume may comprise the volume of all or some of the various liquid compartments of the human body.

In one preferred embodiment, from the electrical data originating from the foot-hand impedance measurements, the device 10 calculates, independently of one another, three volumes of water contained in a user's body:
a volume Vᵢ of intracellular water contained in a user's body,
a volume Vₑ of extracellular water contained in a user's body,
a total volume Vt of water contained in a user's body.

In another preferred embodiment, a volume Vᵢ of intracellular water, a volume Vₑ of extracellular water and a total volume Vt of water are measured independently of one another.

In one embodiment, the total volume Vt of water contained in a patient's body is calculated using the formula Math1.

$$V t = 0.01 * {( \frac{{K}_{b} * {ρ}_{∞} * {H}^{2} * {( \frac{W}{{D}_{b}} )}^{\frac{1}{2}}}{{R}_{∞}} )}^{2 / 3} \qquad [ Math 1 ]$$

where
K_{b} is the shape factor of the patient,
p_{∞} is the equivalent resistivity of the two, extracellular and intracellular, compartments,
H is the height of the patient in cm,
W is the weight in kg,
D_{b} is the density of the patient's body,
R_{∞} is the resistance extrapolated to infinite frequency using the Cole-Cole model.

The value for the equivalent resistivity p_{∞} is obtained using formula Math2.

$${ρ}_{∞} = {( \frac{L B M * 0.732}{1 {0}^{- 2}} )}^{3 / 2} * \frac{{R}_{∞}}{{K}_{b} * {ρ}_{∞} * {H}^{2} * {( \frac{W}{{D}_{b}} )}^{\frac{1}{2}}} \qquad [ Math 2 ]$$

where
LBM is the lean body mass in kilograms.

The value of the shape factor is obtained using formula Math3

$${K}_{b} = \frac{1}{{H}^{2}} * [ ( \frac{{L}_{arm}}{{C}_{arm}^{2}} + \frac{{L}_{trunk}}{{C}_{trunk}^{2}} + \frac{{L}_{leg}}{{C}_{leg}^{2}} ) * ( 2 {L}_{a r m} {C}_{a r m}^{2} + 2 {L}_{trunk} {C}_{trunk}^{2} + 2 {L}_{l e g} {C}_{l e g}^{2} ) ] \qquad [ Math 3 ]$$

where
Lₐᵣₘ, Lₜᵣᵤₙₖ and L_{leg} are, respectively, the length, in centimeters, of the patient's arm, trunk and leg,
Cₐᵣₘ, Cₜᵣᵤₙₖ and C_{leg} are, respectively, the circumference, in centimeters, of the patient's arm, trunk and leg.

The analysis of the electrical data originating from the foot-hand impedance measurements also makes it possible to calculate the user's dry weight, a hyperhydration volume, in liters, and/or a variation in dry weight and/or a variation in hyperhydration.

The analysis of the electrical data originating from the foot-hand impedance measurements and the measurements supplied by the mass sensors 13 also make it possible to calculate a hyperhydration volume, in liters. The hyperhydration corresponds to the surplus of water present in the user's body, this surplus being generated by the malfunctioning of the renal system. As a preference, the precision of the hyperhydration volume is at least to within 0.1 liter.

The analysis of the electrical data originating from the foot-hand impedance measurements and the measurements supplied by the mass sensors 13 also makes it possible to calculate the user's dry weight, in kilograms, preferably with a precision at least to within 0.5 kilogram, or even to within 0.1 kilogram.

Advantageously, the device 10 makes it possible to calculate a variation in hyperhydration, notably a variation in hyperhydration between two dialysis sessions.

In one embodiment, the foot-hand impedance measurements make it possible to determine a resistance (the real part of the impedance) and a reactance (the imaginary part of the impedance). Determining the resistance and the reactance relating to each impedance measurement makes it possible to exploit the impedance measurements using a BIVA (Bioimpedance Vector Analysis) method.

Analysis of the data originating from the photoplethysmographic sensor 23 makes it possible to calculate:
a blood pressure in mmHg or cmHg, and/or
a blood oxygen saturation, as a percentage, with a precision of the order of one decimal place, and/or
the user's heart rate, in beats per minute.

A hand-hand electrocardiogram measurement is performed using the electrodes 21, 22 and 41 which allow the cardiac electrical activity, emitted by the sinus node of the heart which acts as an autonomous electrical power source, to be measured between the user's two hands. In particular, these measurements make it possible to obtain an electrocardiogram of the user.

In one embodiment, the human-machine interface 5 enables the displaying of at least one item of data selected from among:
a user's total mass,
a total volume Vₜ of water contained in a user's body,
a user's dry weight,
a variation in the user's dry weight,
a hyperhydration volume,
a variation in hyperhydration,
a user's heart rate,
a user's blood pressure,
a user's skin temperature,
a visual indication of bioimpedance.

In one embodiment, the medical device further comprises a means, notably wireless, of communication with a portable device comprising a means of communication and a storage means containing anthropometric information about a user including at least one of the items of information that are a date of birth, an ethnicity, height, a gender.

To that end, in one embodiment, the medical device may communicate with an application installed in a user's mobile phone. The storage means may be a memory of the phone, and the application installed in the phone collects the anthropometric data from the phone memory to communicate them to the medical device.

Alternatively, the storage means may be an electronic card belonging to the user, the data recorded in the electronic card being able to be recovered by the medical device directly (if the medical device is able to read the card) or indirectly, via an application installed in the user's phone.

1. A medical device for monitoring biological parameters of a user, comprising:
a plate for measuring a mass of the user,
at least a first electrode and a second electrode for performing impedance measurements at least at two distinct frequencies, and for measuring at least a volume of water in a body of the user, and
a device for measuring blood pressure and/or heart rate.

2. The medical device as claimed in claim 1, wherein the device for measuring blood pressure and/or heart rate is a photoplethysmographic sensor also capable of measuring oxygen saturation of a blood of the user.

3. The medical device as claimed in claim 1, wherein
the plate is equipped with the first electrode able to measure impedance at one of feet of the user,
the second electrode is positioned so as to be able to measure impedance at a first hand of the user, and
impedance measurements comprise at least one measurement of impedance between one of the feet of the user and one of hands of the user.

4. The medical device as claimed in claim 3, wherein
the medical device_comprises a third electrode arranged in contact with a second hand of the user, and
the second and third electrodes are able together to record an electrocardiogram between the first hand and the second hand.

5. The medical device as claimed in claim 1, wherein the medical device comprises a temperature sensor making it possible to measure a temperature of a skin of the user.

6. The medical device as claimed in claim 1, wherein the medical device comprises adjustable means for adjusting a height of the second electrode relative to the plate to adapt a vertical distance, measured between the second electrode and the plate, to suit a height of the user.

7. The medical device as claimed in claim 6, wherein
the medical device comprises a first handgrip, on which the second electrode is fixed, arranged on a rigid arm substantially parallel to the plate, and
the adjustable means is a system for adjusting a height of the rigid arm comprising the first rigid handgrip relative to the plate to enable adjustment of a height of the first rigid handgrip relative to the plate.

8. The medical device as claimed in claim 1, wherein electrodes of the medical device are suited to measuring impedance at a foot or a hand of the user by injecting a current with a strength in a range of from 8 to 200 μA, give or take 10%, and with a frequency sweep extending in a range of from 125 Hz to 500 kHz.

9. The medical device as claimed in claim 1, wherein the impedance measurements comprise at least a first measurement at a low frequency, in a range of from 125 Hz to 5 kHz, or at a medium frequency, in a range of from 5 kHz to 50 kHz, and at least one second measurement at a high frequency, in a range of from 50 kHz to 300 kHz.

10. The medical device as claimed in claim 1, wherein
the medical device comprises an electronic processing unit,
the electrodes and/or the device for measuring blood pressure and/or heart rate, and/or the temperature sensor are connected to the electronic processing unit by a wired or wireless communication device so that the impedance measurements taken using the electrodes are able to be transmitted to the electronic processing unit and so that the electronic processing unit is able to control a current transmitted by the electrodes to a hand or foot of the user.

11. The medical device as claimed in claim 1, wherein the medical device comprises hardware elements and/or software elements configured to obtain, from the impedance measurements between a foot and a hand of the user, measurement of at least one water volume comprising:
a measurement of a volume of intracellular water contained in the body of the user, and
a measurement of a volume of extracellular water contained in the body of the user, and
a measurement of a total volume of water contained in the body of the user.

12. The medical device as claimed in claim 1, wherein the medical device comprises hardware elements and/or software elements configured for performing the following processing operations:
measuring a dry weight,
measuring a hyperhydration volume,
and optionally measuring a variation in dry weight,
and optionally measuring a variation in hyperhydration,
and optionally measuring a blood pressure,
and optionally measuring a pulse wave,
and optionally measuring a heart rate,
and optionally measuring a blood oxygen saturation level,
and optionally measuring a hand-hand electrocardiogram,
and optionally measuring a skin temperature.

13. The medical device as claimed in claim 11, wherein the measurement of the volume of intracellular water, the measurement of the volume of extracellular water, and the measurement of the total volume of water are performed independently of one another.

14. The medical device as claimed in claim 11, wherein the medical device comprises a human-machine interface enabling displaying at least one item of data selected from among:
a total mass of the user,
a total volume of water contained in the body of the user,
a dry weight of the user,
a variation in a dry weight of the user,
a hyperhydration volume,
a variation in hyperhydration,
a heart rate of the user,
a blood pressure of the user,
a skin temperature of the user,
a visual indication of bioimpedance.

15. The medical device as claimed in claim 1, wherein the medical device comprises means of communication with a portable device containing anthropometric information about the user including at least a date of birth, an ethnicity, a height, and a gender.

16. The medical device as claimed in claim 15, wherein the means of communication is a wireless device.

17. The medical device as claimed in claim 1, wherein the two distinct frequencies, are (i) a low or medium frequency, and (ii) a high frequency.

18. The medical device as claimed in claim 1, wherein the at least one measurement of impedance between one of the feet of the user and one of hands of the user is a measurement of impedance between a foot and a hand situated in a same hemicorpus of the body of the user.

19. The medical device as claimed in claim 18, wherein the foot and the hand are situated in the right hemicorpus of the body of the user.

20. The medical device as claimed in claim 9, wherein the impedance measurements comprise at least a third measurement at a third frequency different than the first and second frequencies.
